(19) United States
(12) Patent Application Publication
(10) Pub. No.: US 2024/0200040 A1
(43) Pub. Date: Jun. 20, 2024

(54) CHIMERIC DNA POLYMERASE AND APPLICATION THEREOF

(71) Applicant: BGI SHENZHEN, Shenzhen, Guangdong (CN)

(72) Inventors: Qun AN, Shenzhen, Guangdong (CN); Miaomiao GUO, Shenzhen, Guangdong (CN); Feng XI, Shenzhen, Guangdong (CN); Fei GUO, Shenzhen, Guangdong (CN); Yue ZHENG, Shenzhen, Guangdong (CN); Yuliang DONG, Shenzhen, Guangdong (CN); Wenwei ZHANG, Shenzhen, Guangdong (CN)

(21) Appl. No.: 17/787,955

(22) PCT Filed: Dec. 23, 2019
(86) PCT No.: PCT/CN2019/127462

§ 371 Date: Jun. 22, 2022

(51) Int. Cl. C12N 9/12; C12P 19/34; C12Q 1/48

(52) U.S. Cl. CPC C12N 9/1252; C12P 19/34; C12Q 1/485; C12Y 207/07007

(57) ABSTRACT

A chimeric DNA polymerase includes: a first fragment having at least 80% homology to at least part of an N-end domain of KOD DNA polymerase; a second fragment having at least 80% homology to at least part of an exonucleolytic domain of Pab DNA polymerase; a third fragment having at least 80% homology to at least part of the N-end domain of KOD DNA polymerase; a fourth fragment having at least 80% homology to at least part of a palm domain of Pfu DNA polymerase; a fifth fragment having at least 80% homology to at least part of a finger domain of Pab DNA polymerase; a sixth fragment having at least 80% homology to at least part of the palm domain of Pfu DNA polymerase; and a seventh fragment having at least 80% homology to at least part of a thumb domain of KOD DNA polymerase.

### INCORPORATION BY REFERENCE

The ASCII plain text file entitled 980P002_ST25.txt dated Oct. 24, 2023 and having a size of 40,000 bytes containing a Sequence Listing is hereby incorporated by reference.

### FIELD

The present disclosure relates to the field of biology. In particular, the present disclosure relates to a chimeric DNA polymerase and application thereof.

### BACKGROUND

DNA polymerases are enzymes for replicating and synthesizing a new DNA strand complementary to the sequence of the template from a 5′-end by using a single DNA strand as a template and four deoxynucleotides as substrates. The DNA polymerase can add free sqnucleotides to an 3′-end of the newly formed strand to elongate the new strand in a direction from 5′-end to 3′-end. Certain enzymes with 3′-to-5′ exoribonuclease activity can correct errors in the newly synthesized DNA. During PCR amplification, the enzymes can cut a mismatched base, when it is produced, and thereafter, the polymerase can re-insert a correct base and continue the replication, thereby ensuring the accuracy of amplification. The enzymes with the correcting function have a lower error rate than ordinary DNA polymerases (such as Taq DNA polymerase), and are suitable for experiments that require high PCR fidelity, for example, gene screening, sequencing, mutation detection, etc.

DNA polymerases are mainly divided into six families: A, B, C, D, X, and Y. At present, the known thermostable DNA polymerases all belong to the family A or the family B. DNA polymerases belonging to the family A are all derived from eubacteria, for example, Thermus aquaticus (Taq), Thermus thermophilus (Tth), Thermus caldophilus (Tca), Thermus flavus (Tfl), and Thermus filiformis (Tfi) from the genus Thermus, and Bacillus stearothemophilis (Bst) from the genus Bacillus. The thermostable DNA polymerases belonging to the family B are all derived from archaea, for example, Thermococcus litoralis (Tli) from the genus Thermococcus, Pyrococcus furiosus (Pfu), Thermococcus kodacaraensis (KOD1), Pyrococcus woesei (Pwo), Thermococcus gorgonarius (Tgo) and Pyrococcus abyssi (Pab) from the genus Thermococcu, etc.

The amino acid sequence of a polymerase is the basis for the functional structure of the polymerase. Based on the structure-function analysis of a DNA polymerase, the various functions of the DNA polymerase, such as catalytic activity, correcting, nucleotide transfer, substrate binding, etc., have been assigned to various domains. As an example, the archaeal DNA polymerase is usually divided into five domains, i.e., an N-end domain, an exonucleolytic domain, a palm domain, a finger domain, and a thumb domain. It is generally believed that the activity of DNA polymerase involves the palm domain, the finger domain and the thumb domain. The palm domain is a catalytic site of the polymerase. The thumb domain interacts with newly synthesized dsDNA and interacts with the introduced nucleotides. The finger domain plays a role in the template immobilization and nucleotide specificity. The exonucleolytic domain is associated with either 5′-to-3′ exonucleolytic activity or 3′-to-5′ exonucleolytic activity or associated with both activities, and thus it is used to remove erroneously inserted bases. The domains of the DNA polymerase cooperate closely with each other to complete the whole process of DNA amplification.

However, it is necessary to further study the current DNA polymerases.

### SUMMARY

The present disclosure aims to solve at least one of the technical problems existing in the prior art to a certain extent. To this end, the present disclosure provides a chimeric DNA polymerase, a method for obtaining the chimeric DNA polymerase, an isolated nucleic acid, a construct, a recombinant cell or recombinant microorganism, a kit, and applications thereof. The chimeric DNA polymerase has various properties such as high processivity and elongation rate, thermal stability, strong resistance to salts, high fidelity, etc., and thus they can meet the requirements of DNA amplification, synthesis, detection, sequencing, etc., having a wide application prospect.

It should be noted that the present disclosure is based on Applicant's following findings.

Currently, the advantages of DNA polymerases with the correcting function are offset by their relatively low processivity, which may result in a reduced yield of DNA amplification products. Taq DNA polymerase, as a representative of the DNA polymerases in the family A, has a relatively high amplification efficiency but lacks of fidelity. KOD/Pfu DNA polymerases, as representatives of the DNA polymerases in the family B, have disadvantages in that the high processivity is accompanied with the high fidelity.

In view of this, Applicant combined heterologous domains from different DNA polymerases to form a chimeric DNA polymerase, in order to form a specific spatial structure and exhibit corresponding functional characteristics by utilizing the unique interactions within each of different heterologous domains and between the different heterologous domains when these domains are fused. Specifically, Applicant studied 7 types of DNA polymerases, i.e., Pyrococcus furiosus (Pfu), Thermococcus kodacaraensis (KOD), Pyrococcus woesei (Pwo), Thermococcus gorgonarius (Tgo), Pyrococcus abyssi (Pab), Pyrococcus species GB-D (Deep vent), and Thermococcus sp. 9°N-7 (9°N), and 5 types of domains of each one of these DNA polymerases, to select chimeric DNA polymerases having the performances such as high processivity, high elongation rate, thermal stability, resistance to salts, and high fidelity, etc., which can meet the requirements of DNA amplification, synthesis, detection, sequencing, etc., thereby having a wide application prospect.

To this end, in an aspect of the present disclosure, the present disclosure provides a chimeric DNA polymerase. According to an embodiment of the present disclosure, the chimeric DNA polymerase includes: a first peptide fragment having at least 80% homology to at least one part of an amino acid sequence in an N-end domain of a KOD DNA polymerase; a second peptide fragment having at least 80% homology to at least one part of an amino acid sequence in an exonucleolytic domain of a Pab DNA polymerase, an N-end of the second peptide fragment being connected to a C-end of the first peptide fragment; a third peptide fragment having at least 80% homology to at least part of amino acids in the N-end domain of the KOD DNA polymerase, an N-end of the third peptide fragment being connected to a C-end of the second peptide fragment; a fourth peptide fragment having at least 80% homology to at least part of amino acids in a palm domain of a Pfu DNA polymerase, an N-end of the fourth peptide fragment being connected to a C-end of the third peptide fragment; a fifth peptide fragment having at least 80% homology to at least part of amino acids in a finger domain of the Pab DNA polymerase, an N-end of the fifth peptide fragment being connected to a C-end of the fourth peptide fragment; a sixth peptide fragment having at least 80% homology to at least part of the amino acids in the palm domain of the Pfu DNA polymerase, an N-end of the sixth peptide fragment being connected to a C-end of the fifth peptide fragment; and a seventh peptide fragment having at least 80% homology to at least part of amino acids in a thumb domain of the KOD DNA polymerase, an N-end of the seventh peptide fragment being connected to a C-end of the sixth peptide fragment.

Based on 7 types of DNA polymerases, i.e., Pyrococcus furiosus (Pfu), Thermococcus kodacaraensis (KOD), Pyrococcus woesei (Pwo), Thermococcus gorgonarius (Tgo), Pyrococcus abyssi (Pab), Pyrococcus species GB-D (Deep vent), and Thermococcus sp. 9°N-7 (9° N), and 5 types of domains of each of these DNA polymerases, Applicant has screened out the chimeric DNA polymerases having the performances such as high processivity, high elongation rate, thermal stability, resistance to salts, and high fidelity, etc., which can meet the requirement of DNA amplification, synthesis, detection, sequencing, etc., thereby having a wide application prospect.

According to an embodiment of the present disclosure, the chimeric DNA polymerase may further have the following additional technical features.

According to an embodiment of the present disclosure, the first peptide fragment has at least 80% homology to an amino acid sequence of sites 1 to 130 of the KOD DNA polymerase.

According to an embodiment of the present disclosure, the second peptide fragment has at least 80% homology to an amino acid sequence of sites 131 to 337 of the Pab DNA polymerase. According to an embodiment of the present disclosure, the third peptide fragment has at least 80% homology to an amino acid sequence of sites 338 to 373 of the KOD DNA polymerase.

According to an embodiment of the present disclosure, the fourth peptide fragment has at least 80% homology to an amino acid sequence of sites 374 to 448 of the Pfu DNA polymerase.

According to an embodiment of the present disclosure, the fifth peptide fragment has at least 80% homology to an amino acid sequence of sites 449 to 500 of the Pab DNA polymerase.

According to an embodiment of the present disclosure, the sixth peptide fragment has at least 80% homology to an amino acid sequence of sites 501 to 591 of the Pfu DNA polymerase.

According to an embodiment of the present disclosure, the seventh peptide fragment has at least 80% homology to an amino acid sequence of sites 591 to 774 of the KOD DNA polymerase.

According to an embodiment of the present disclosure, the chimeric DNA polymerase has an amino acid sequence set forth as SEQ ID NO: 1.

In another aspect of the present disclosure, the present disclosure provides an isolated nucleic acid. According to an embodiment of the present disclosure, the isolated nucleic acid encodes the aforementioned chimeric DNA polymerase. Therefore, the isolated nucleic acid according to the embodiment of the present disclosure can be used to encode the chimeric DNA polymerases having the performances such as high processivity, high elongation rate, thermal stability, strong resistance to salts, and high fidelity, etc., which can meet the requirements of DNA amplification, synthesis, detection, sequencing, etc., thereby having a wide application prospect.

According to an embodiment of the present disclosure, the isolated nucleic acid has a nucleotide sequence set forth as SEQ ID NO: 2.

In yet another aspect of the present disclosure, the present disclosure provides a construct. According to an embodiment of the present disclosure, the construct contains the isolated nucleic acid as described above. Thus, the construct according to the embodiment of the present disclosure can express the chimeric DNA polymerases having all the performances such as high processivity, high elongation rate, thermal stability, strong resistance to salts, and high fidelity, etc., which can meet the requirements of DNA amplification, synthesis, detection, sequencing, etc., thereby having a wide application prospect.

In yet another aspect of the present disclosure, the present disclosure provides a recombinant cell or recombinant microorganism. According to an embodiment of the present disclosure, the recombinant cell or recombinant microorganism contains the isolated nucleic acid as described above. Thus, the recombinant cell or recombinant microorganism according to the embodiment of the present disclosure can express the chimeric DNA polymerases all the performances such as high processivity, high elongation rate, thermal stability, strong resistance to salts, and high fidelity, etc., which can meet the requirements of DNA amplification, synthesis, detection, sequencing, etc., thereby having a wide application prospect.

In yet another aspect of the present disclosure, the present disclosure provides a method for obtaining the aforementioned chimeric DNA polymerase. According to an embodiment of the present disclosure, the method comprises: culturing the aforementioned recombinant cell or recombinant microorganism under conditions suitable for expressing the chimeric DNA polymerase, to obtain the chimeric DNA polymerase. Thus, the method according to the embodiment of the present disclosure can obtain the chimeric DNA polymerase all the performances such as high processivity, high elongation rate, thermal stability, strong resistance to salts, and high fidelity, etc., which can meet the requirements of DNA amplification, synthesis, detection, sequencing, etc., thereby having a wide application prospect.

In yet another aspect of the present disclosure, the present disclosure provides a kit. According to an embodiment of the present disclosure, the kit contains the aforementioned chimeric DNA polymerase, isolated nucleic acid, construct, recombinant cell, or recombinant microorganism. Therefore, the use of the kit according to the embodiment of the present disclosure to amplify DNA has the advantages such as high amplification product yield and high amplification accuracy, and is suitable for massive production and wide application.

In yet another aspect of the present disclosure, the present disclosure provides uses of the aforementioned chimeric DNA polymerase, isolated nucleic acid, construct, recombinant cell or recombinant microorganism, or kit in DNA amplification. Thus, the DNA amplification has the advantages such as high amplification product yield and high amplification accuracy, and is suitable for wide production and application.

According to an embodiment of the present disclosure, the chimeric DNA polymerase, the isolated nucleic acid, the construct, the recombinant cell or recombinant microorganism, or the kit is used for gene screening, sequencing or mutation detection.

Additional aspects and advantages of the present disclosure will be in part provided in the following description, or they will be become apparent from the following description, or they can be learned by practice of the present disclosure.

### BRIEF DESCRIPTION OF DRAWINGS

The above and/or additional aspects and advantages of the present disclosure will become apparent and readily understood from the following description of embodiments in conjunction with the accompanying drawings, in which:

FIG. 1 is a schematic structural diagram of a chimeric DNA polymerase according to an embodiment of the present disclosure;

FIG. 2 is an electropherogram of enzyme expression and purification according to an embodiment of the present disclosure;

FIG. 3 is a comparison electropherogram of amplification performances of fragments with different amplification lengths according to an embodiment of the present disclosure;

FIG. 4 is an electropherogram of thermal stability comparison according to an embodiment of the present disclosure; and

FIG. 5 is a comparison electropherogram of amplifications with different templates according to an embodiment of the present disclosure.

### DESCRIPTION OF EMBODIMENTS

Embodiments of the present disclosure are described in detail below. The embodiments described below are merely illustrative for explaining the present disclosure, and they should not be construed as limiting the present disclosure.

It should be noted that the terms “first” and “second” are only used for descriptive purposes, and cannot be understood as indicating or implying relative importance or implying the number of indicated technical features. Thus, a feature defined by “first” or “second” may expressly or implicitly include one or more of the features. Further, in the description of the present disclosure, “plurality” means two or more, unless otherwise specified.

The present disclosure provides a chimeric DNA polymerase, a method for obtaining the chimeric DNA polymerase, an isolated nucleic acid, a construct, a recombinant cell or recombinant microorganism, a kit, and applications thereof, which will be described in detail below.

### Chimeric DNA Polymerase

In one aspect of the present disclosure, the present disclosure provides a chimeric DNA polymerase. According to an embodiment of the present disclosure, the chimeric DNA polymerase incudes: a first peptide fragment having at least 80% homology to at least one part of an amino acid sequence in an N-end domain of a KOD DNA polymerase; a second peptide fragment having at least 80% homology to at least one part of an amino acid sequence in an exonucleolytic domain of a Pab DNA polymerase, an N-end of the second peptide fragment being connected to a C-end of the first peptide fragment; a third peptide fragment having at least 80% homology to at least part of amino acids in the N-end domain of the KOD DNA polymerase, an N-end of the third peptide fragment being connected to a C-end of the second peptide fragment; a fourth peptide fragment having at least 80% homology to at least part of amino acids in a palm domain of a Pfu DNA polymerase, an N-end of the fourth peptide fragment being connected to a C-end of the third peptide fragment; a fifth peptide fragment having at least 80% homology to at least part of amino acids in a finger domain of the Pab DNA polymerase, an N-end of the fifth peptide fragment being connected to a C-end of the fourth peptide fragment; a sixth peptide fragment having at least 80% homology to at least part of the amino acids in the palm domain of the Pfu DNA polymerase, an N-end of the sixth peptide fragment being connected to a C-end of the fifth peptide fragment; and a seventh peptide fragment having at least 80% homology to at least part of amino acids in a thumb domain of the KOD DNA polymerase, an N-end of the seventh peptide fragment being connected to a C-end of the sixth peptide fragment.

In order to develop a DNA polymerase with high processivity and improved fidelity, Applicant focused on the DNA polymerases with thermostability from the family A and the family B among 6 types of DNA polymerases, analyzed the amplification performance characteristics of each enzyme, and selected chimeric candidates. Based on polymerase structure analysis, sequence analysis, and taking the requirement on the amplification fidelity into consideration, novel chimeric DNA polymerases with potential functions were screened through different chimerism of amino acid sequences of various domains of 2, 3, 4 or 5 types of enzymes, containing the amino acid sequences of corresponding domains of the following 7 DNA polymerases. These DNA polymerases are all DNA polymerases of the family B of archaea, derived from Pyrococcus furiosus (Pfu), Thermococcus kodacaraensis (KOD), Pyrococcus woesei (Pwo), Thermococcus gorgonarius (Tgo), Pyrococcus abyssi (Pab), Pyrococcus species GB-D (Deep vent), and Thermococcus sp. 9°N-7 (9°N), respectively. The above-mentioned 7 types of DNA polymerases each having 5 domains provide different combinations of domains. That is, there are 7 candidates for each domain of the chimeric polymerase, and accordingly, a total of (5⁷-7) chimeric forms for the possible chimeric polymerases.

In order to further narrow the above chimeric library, the performances thereof are analyzed as follow for optimization analysis. The performance characteristics of the above 7 types of DNA polymerases were compared and analyzed. For example, the DNA polymerases, Pfu and Pab, have better fidelity performance than other enzymes. Therefore, the chimeric combinations whose exonucleolytic domains are the corresponding nucleoside sequence of these two DNA polymerases are preferred.

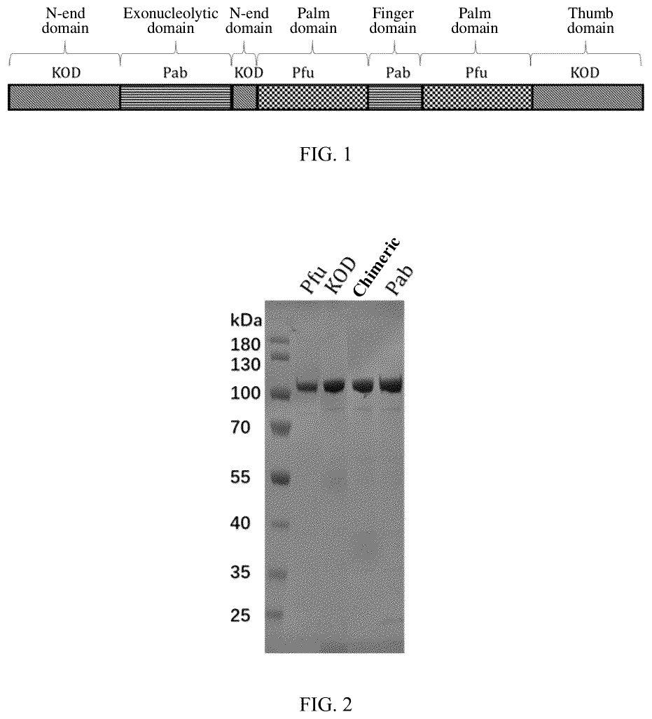

Similarly, the candidate sequences, which are suitable as the palm, finger, thumb and N-end domains, are screened based on the research and analysis of the amplification performance and thermal stability of the respective enzymes, in order to further reduce the chimeric combinations. Through the above screening, the corresponding nucleotide sequences were obtained based on the amino acid sequences of the candidate chimeric polymerases, and constructed into expression vectors. The expression bacteria are induced to express the candidate chimeric polymerases, and the expressed candidate chimeric polymerases are then purified. The expression performance, fidelity, thermal stability and synthesis capability of the chimeric enzymes were compared with each other, in order to select the optimal chimeric DNA polymerase of the present disclosure. The chimeric DNA polymerase having the structure as illustrated in FIG. 1 has high processivity, high elongation rate, thermal stability, strong resistance to salts, and high fidelity, which can meet the requirements of DNA amplification, synthesis, detection, and sequencing, etc., thereby having a broad application prospect.

Amino acid sequence of Pab DNA polymerase (SEQ ID NO: 3):
MIIDADYITEDGKPIIRIFKKEKGEFKVEYDRTFRPYIYALLKDDSAIDEVKKITAERHG
KIVRITEVEKVQKKFLGRPIEVWKLYLEHPQDVPAIREKIREHPAVVDIFEYDIPFAKRY
LIDKGLTPMEGNEELTFLAVDIETLYHEGEEFGKGPIIMISYADEEGAKVITWKSID
LPYVEVVSSEREMIKRLVKVIREKDPDVIITYNGDNFDFPYLLKRAEKLGIKLPL
GRDNSEPKMQRMGDSLAVEIKGRIHFDLFPVIRRTINLPTYTLEAVYEAIFGKSK
EKVYAHEIAEAWETGKGLERVAKYSMEDAKVTFELGKEFFPMEAQLARLVGQP
VWDVSRSSTGNLVEWFLLRKAYERNELAPNKPDEREYERRLRESYEGGYVKEPEKG
LWEGIVSLDFRSLYPSIIITHNVSPDTLNRENCKEYDVAPQVGHRFCKDFPGFIPSLLGN
LLEERQKIKKRMKESKDPVEKKLLDYRQRAIKILANSYYGYYGYAKARWYCKE
CAESVTAWGRQYIDLVRRELESRGFKVLYIDTDGLYATIPGAKHEEIKEKALKFVEYIN
SKLPGLLELEYEGFYARGFFVTKKKYALIDEEGKIVTRGLEIVRRDWSEIAKETQAKV
LEAILKHGNVDEAVKIVKEVTEKLSKYEIPPEKLVIYEQITRPLSEYKAIGPHVAVAKRL
AAKGVKVKPGMVIGYIVLRGDGPISKRAIAIEEFDPKKHKYDAEYYIENQVLPAVERI
LRAFGYRKEDLKYQKTKQVGLGAWLKF
Amino acid sequence of Pfu DNA polymerase (SEQ ID NO: 4):
MILDVDYITEEGKPVIRLFKKENGKFKIEHDRTFRPYIYALLRDDSKIEEVKKITGERH
GKIVRIVDVEKVEKKFLGKPITVWKLYLEHPQDVPTLREKVREHPAVVDIFEYDIPFA
KRYLIDKGLIPMEGEEELKILAFDIETLYHEGEEFGKGPIIMISYADENEARVITWKNID
LPYVESVSTEKEMIKRFLRIIREKDPDIIVTYNGDSFDFPYLAKRAEKLGIKLTIGRDGS
EPKMQRIGDMTAVEVKGRIHFDLYHVIRTTINLPTYTLEAVYEAIFGKPKEKVYADEI
AKAWESGENLERVAKYSMEDAKATYELGKEFLPMEIQLSRLVGQPLWDVSRSSTGN
LVEWFLLRKAYERNEVAPNKPSEEEYQRRLRESYTGGFVKEPEKGLWENIVYLD
YKSLYPSIIITHNVSPDTLNLEGCKNYDIAPQVGHKFCKDIPGFIPSLLGHLLEER
QKIKTKMKETQDPIEKILLDYRQKAIKLLANSFYGYYGYAKARWYCKECAESV
TAWGRKYIELVWKELEEKFGFKVLYIDTDGLYATIPGGESEEIKKKALEFVKYI
NSKLPGLLELEYEGFYKRGFFVTKKRYAVIDEEGKVITRGLEIVRRDWSEIAKETQ
ARVLETILKHGDVEEAVRIVKEVIQKLANYEIPPEKLAIYEQITRPLHEYKAIGPHVAV
AKKLAAKGVKIKPGMVIGYIVLRGDGPISNRAILAEEYDPKKHKYDAEYYIENQVLP
AVLRILEGFGYRKEDLRYQKTRQVGLTSWLNIKKS
Amino acid sequence of KOD DNA polymerase (SEQ ID NO: 5):
MILDTDYITEDGKPVIRIFKKENGEFKIEYDRTFEPYFYALLKDDSAIEEVKKITA
ERHGTVVTVKRVEKVQKKFLGRPVEVWKLYFTHPQDVPAIRDKIREHPAVIDIY
EYDIPFAKRYLIDKGLVPMEGDEELKMLAFDIETLYHEGEEFAEGPILMISYADEEGA
RVITWKNVDLPYVDVVSTEREMIKRFLRVVKEKDPDVLITYNGDNFDFAYLKKRCEK
LGINFALGRDGSEPKIQRMGDRFAVEVKGRIHFDLYPVIRRTINLPTYTLEAVYEAVFG
QPKEKVYAEEITTAWETGENLERVARYSMEDAKVTYELGKEFLPMEAQLSRLIGQSL
WDVSRSSTGNLVEWFLLRKAYERNELAPNKPDEKELARRRQSYEGGYVKEPERGLW
ENIVYLDFRSLYPSIIITHNVSPDTLNREGCKEYDVAPQVGHRFCKDFPGFIPSLLGDLL
EERQKIKKKMKATIDPIERKLLDYRQRAIKILANSYYGYYGYARARWYCKECAESVT
AWGREYITMTIKEIEEKYGFKVIYSDTDGFFATIPGADAETVKKKAMEFLKYINAKLP
GALELEYEGFYKRGFFVTKKKYAVIDEEGKITTRGLEIVRRDWSEIAKETQARVLE
ALLKDGDVEKAVRIVKEVTEKLSKYEVPPEKLVIHEQITRDLKDYKATGPHVAVA
KRLAARGVKIRPGTVISYIVLKGSGRIGDRAIPFDEFDPTKHKYDAEYYIENQVL
PAVERILRAFGYRKEDLRYQKTRQVGLSAWLKPKGT

According to an embodiment of the present disclosure, the first peptide fragment has at least 80% homology to an amino acid sequence of sites 1 to 130 of the KOD DNA polymerase. The amino acid sequence of sites 1 to 130 of the KOD DNA polymerase is a first gray-marked sequence fragment in the sequence set forth as SEQ ID NO: 5, i.e., a partial sequence in the N-end domain of the KOD DNA polymerase.

According to an embodiment of the present disclosure, the second peptide fragment has at least 80% homology to an amino acid sequence of sites 131 to 337 of the Pab DNA polymerase. The amino acid sequence of sites 131 to 337 of the Pab DNA polymerase is a first gray-marked sequence fragment in the sequence set forth as SEQ ID NO: 3, i.e., a partial sequence in the exonucleolytic domain of the Pab DNA polymerase.

According to an embodiment of the present disclosure, the third peptide fragment has at least 80% homology to an amino acid sequence of sites 338 to 373 of the KOD DNA polymerase. The amino acid sequence of sites 338 to 373 of the KOD DNA polymerase is a second gray-marked sequence fragment in a sequence set forth as SEQ ID NO: 5, i.e., a partial sequence in the N-end domain of the KOD DNA polymerase.

According to an embodiment of the present disclosure, the fourth peptide fragment has at least 80% homology to an amino acid sequence of sites 374 to 448 of the Pfu DNA polymerase. The amino acid sequence of sites 374 to 448 of the Pfu DNA polymerase is a first gray-marked sequence fragment in the sequence set forth as SEQ ID NO: 4, i.e., a partial sequence in the palm domain of the Pfu DNA polymerase.

According to an embodiment of the present disclosure, the fifth peptide fragment has at least 80% homology to an amino acid sequence of sites 449 to 500 of the Pab DNA polymerase. The amino acid sequence of sites 449 to 500 of the Pab DNA polymerase is a second gray-marked sequence fragment in the sequence set forth as SEQ ID NO: 3, i.e., a partial sequence in the finger domain of the Pab DNA polymerase.

According to an embodiment of the present disclosure, the sixth peptide fragment has at least 80% homology to an amino acid sequence of sites 501 to 591 of the Pfu DNA polymerase. The amino acid sequence of sites 501 to 591 of the Pfu DNA polymerase is a second gray-marked sequence fragment in the sequence set forth as SEQ ID NO: 4, i.e., a partial sequence in the palm domain of the Pfu DNA polymerase.

According to an embodiment of the present disclosure, the seventh peptide fragment has at least 80% homology to an amino acid sequence of sites 591 to 774 of the KOD DNA polymerase. The amino acid sequence of sites 591 to 774 of the KOD DNA polymerase is a third gray-marked sequence in the sequence set forth as SEQ ID NO: 5, i.e., a partial sequence in the thumb domain of the KOD DNA polymerase.

As a result, the obtained chimeric DNA polymerases have the properties such as high processivity, high elongation rate, thermal stability, strong resistance to salts, and high fidelity, and thus they can meet the requirements of DNA amplification, synthesis, detection, sequencing, etc., thereby having a broad application prospect.

According to an embodiment of the present disclosure, the chimeric DNA polymerase has the amino acid sequence set forth as SEQ ID NO: 1. In this way, the chimeric DNA polymerase according to the embodiment of the present disclosure has the properties such as high processivity, high elongation rate, thermal stability, strong resistance to salts, and high fidelity, and it can meet the requirements of DNA amplification, synthesis, detection, sequencing, etc., thereby having a broad application prospect.

(SEQ ID NO: 1)
MILDTDYITEDGKPVIRIFKKENGEFKIEYDRTFEPYFYALLKDDSAIE
EVKKITAERHGTVVTVKRVEKVQKKFLGRPVEVWKLYFTHPQDVPAIRD
KIREHPAVIDIYEYDIPFAKRYLIDKGLVPMEGNEELTFLAVDIETLYH
EGEEFGKGPIIMISYADEEGAKVITWKSIDLPYVEVVSSEREMIKRLVK
VIREKDPDVIITYNGDNFDFPYLLKRAEKLGIKLPLGRDNSEPKMQRMG
DSLAVEIKGRIHFDLFPVIRRTINLPTYTLEAVYEAIFGKSKEKVYAHE
IAEAWETGKGLERVAKYSMEDAKVTFELGKEFFPMEAQLARLVGQSLWD
VSRSSTGNLVEWFLLRKAYERNELAPNKPDEEEYQRRLRESYTGGFVKE
PEKGLWENIVYLDYKSLYPSIIITHNVSPDTLNLEGCKNYDIAPQVGHK
FCKDIPGFIPSLLGNLLEERQKIKKRMKESKDPVEKKLLDYRQRAIKIL
ANSYYGYYGYAKARWYCKECAESVTAWGRKYIELVWKELEEKFGFKVLY
IDTDGLYATIPGGESEEIKKKALEFVKYINSKLPGLLELEYEGFYKRGF
FVTKKKYAVIDEEGKITTRGLEIVRRDWSEIAKETQARVLEALLKDGDV
EKAVRIVKEVTEKLSKYEVPPEKLVIHEQITRDLKDYKATGPHVAVAKR
LAARGVKIRPGTVISYIVLKGSGRIGDRAIPFDEFDPTKHKYDAEYYIE
NQVLPAVERILRAFGYRKEDLRYQKTRQVGLSAWLKPKGT

### Isolated Nucleic Acid

In another aspect of the present disclosure, the present disclosure provides an isolated nucleic acid. According to an embodiment of the present disclosure, the isolated nucleic acid encodes the aforementioned chimeric DNA polymerase. Thus, the isolated nucleic acid according to the embodiment of the present disclosure can be used to encode the chimeric DNA polymerase having the properties such as high processivity, high elongation rate, thermal stability, strong resistance to salts, and high fidelity, which can meet the requirements of DNA amplification, synthesis, detection, sequencing, etc., thereby having a broad application prospect.

According to an embodiment of the present disclosure, a nucleic acid encoding the first peptide fragment has at least 80% homology to a nucleotide sequence of sites 1 to 390 of the KOD DNA polymerase. The sequence of sites 1 to 390 of the nucleotide sequence encoding the KOD DNA polymerase is a first gray-marked sequence fragment in a sequence set forth as SEQ ID NO: 8, i.e., a partial sequence encoding the N-end domain of the encoding KOD DNA polymerase.

According to an embodiment of the present disclosure, a nucleic acid encoding the second peptide fragment has at least 80% homology to a nucleotide sequence of sites 391 to 1011 of the Pab DNA polymerase. The sequence of sites 391 to 1011 of a nucleotide sequence encoding the Pab DNA polymerase is a first gray-marked sequence fragment in a sequence set forth as SEQ ID NO: 6, i.e., a partial sequence encoding the exonucleolytic domain of the Pab DNA polymerase.

According to an embodiment of the present disclosure, a nucleic acid encoding the third peptide fragment has at least 80% homology to a nucleotide sequence at sites 1012 to 1119 of the KOD DNA polymerase. The sequence of sites 1012 to 1119 of the nucleotide sequence encoding the KOD DNA polymerase is a second gray-marked sequence fragment in the sequence set forth as SEQ ID NO: 8, i.e., a partial sequence encoding the N-end domain of the encoding KOD DNA polymerase.

According to an embodiment of the present disclosure, a nucleic acid encoding the fourth peptide fragment has at least 80% homology to a nucleotide sequence of sites 1120 to 1344 of the Pfu DNA polymerase. The sequence of sites 1120 to 1344 of a nucleotide sequence encoding the Pfu DNA polymerase is a first gray-marked sequence fragment in a sequence set forth as SEQ ID NO: 7, i.e., a partial sequence encoding the palm domain of the Pfu DNA polymerase.

According to an embodiment of the present disclosure, a nucleic acid encoding the fifth peptide fragment has at least 80% homology to a nucleotide sequence at sites 1345 to 1500 of the Pab DNA polymerase. The sequence of sites 1345 to 1500 of the nucleotide sequence encoding the Pab DNA polymerase is a second gray-marked sequence in the sequence set forth as SEQ ID NO: 6, i.e., a partial sequence encoding the finger domain of the Pab DNA polymerase.

According to an embodiment of the present disclosure, a nucleic acid encoding the sixth peptide fragment has at least 80% homology to a nucleotide sequence of sites 1501 to 1773 of the Pfu DNA polymerase. The sequence of sites 1501 to 1773 of the nucleotide sequence encoding the Pfu DNA polymerase is a second gray-marked sequence in the sequence set forth as SEQ ID NO: 7, i.e., a partial sequence encoding the palm domain of the Pfu DNA polymerase.

According to an embodiment of the present disclosure, a nucleic acid encoding the seventh peptide fragment has at least 80% homology to a nucleotide sequence of sites 1771 to 2325 of the KOD DNA polymerase. The sequence of sites 1771 to 2325 of the nucleotide sequence encoding the KOD DNA polymerase is a third gray-marked sequence in the sequence set forth as SEQ ID NO: 8, i.e., a partial sequence encoding the thumb domain of KOD DNA polymerase.

As a result, the obtained chimeric DNA polymerase has the properties such as high processivity, high elongation rate, thermal stability, strong resistance to salts, and high fidelity, and thus it can meet the requirements of DNA amplification, synthesis, detection, sequencing, etc., thereby having a broad application prospect.

According to an embodiment of the present disclosure, the isolated nucleic acid has a nucleotide sequence set forth as SEQ ID NO:2.

(SEQ ID NO: 2)
ATGATTCTGGACACCGATTACATCACCGAAGATGGCAAGCCAGTTATCCGCATTTT
CAAAAAAGAGAATGGTGAATTCAAGATCGAATATGATCGTACCTTCGAGCCGTACT
TCTATGCTCTGCTGAAAGACGATAGCGCGATTGAGGAGGTCAAGAAAATCACCGC
GGAGCGTCACGGTACGGTTGTTACCGTGAAACGCGTGGAGAAAGTCCAGAAGAA
ATTTCTGGGTCGCCCGGTTGAAGTGTGGAAGCTGTACTTTACGCATCCGCAAGATG
TTCCGGCGATTCGCGATAAGATTCGTGAGCACCCGGCAGTCATTGACATCTACGAG
TATGACATTCCGTTCGCCAAGCGTTATCTGATCGATAAGGGTCTGGTCCCGATGGA
GGGGAACGAGGAGCTAACGTTTCTAGCCGTTGATATAGAAACATTGTACCATGAA
GGAGAGGAGTTCGGGAAAGGGCCAATAATAATGATCAGCTACGCCGACGAGGAA
GGGGCCAAGGTGATAACTTGGAAGAGCATAGACTTACCTTACGTTGAAGTGGTTT
CGAGCGAGAGGGAGATGATAAAGAGGCTCGTGAAGGTAATTAGAGAGAAAGATC
CCGACGTGATAATAACGTACAATGGTGATAATTTCGACTTTCCGTACCTCTTAAAGA
GGGCTGAAAAGCTCGGAATAAAGCTCCCCCTTGGAAGGGACAATAGCGAGCCGA
AAATGCAGAGGATGGGGGATTCATTAGCCGTAGAGATAAAGGGCAGAATACACTT
CGATTTATTCCCCGTCATAAGAAGAACGATCAACCTTCCAACATACACCCTCGAAG
CGGTTTATGAGGCTATATTTGGAAAGTCTAAGGAGAAAGTCTATGCCCATGAGATA
GCTGAGGCCTGGGAAACCGGGAAAGGGCTAGAGAGGGTAGCTAAGTATTCAATG
GAAGATGCGAAGGTAACCTTTGAGCTCGGAAAGGAGTTCTTCCCGATGGAAGCCC
AGCTAGCTAGGCTCGTTGGCCAAAGCCTGTGGGACGTTAGCCGCAGCAGCACCGG
TAACTTAGTTGAATGGTTCTTGCTGCGTAAGGCATACGAACGCAATGAGCTGGCGC
CGAACAAACCGGACGAAGAGGAGTATCAAAGAAGGCTCAGGGAGAGCTACACA
GGTGGATTCGTTAAAGAGCCAGAAAAGGGGTTGTGGGAAAACATAGTATACCTAG
ATTACAAATCACTATATCCCTCGATTATAATTACCCACAATGTTTCTCCCGATACTCT
AAATCTTGAGGGATGCAAGAACTATGATATCGCTCCTCAAGTAGGCCACAAGTTCT
GCAAGGACATCCCTGGTTTCATACCAAGCTTACTGGGTAACCTACTGGAGGAGAG
ACAAAAGATAAAAAAGAGAATGAAAGAAAGTAAAGATCCCGTCGAGAAGAAACT
CCTTGATTACAGACAGAGAGCTATAAAAATACTTGCAAACAGCTATTATGGCTATTA
TGGATATGCAAAAGCAAGATGGTACTGTAAGGAGTGTGCTGAGAGCGTTACTGCC
TGGGGAAGAAAGTACATCGAGTTAGTATGGAAGGAGCTCGAAGAAAAGTTTGGAT
TTAAAGTCCTCTACATTGACACTGATGGTCTCTATGCAACTATCCCAGGAGGAGAA
AGTGAGGAAATAAAGAAAAAGGCTCTAGAATTTGTAAAATACATAAATTCAAAGC
TCCCTGGACTGCTAGAGCTTGAATATGAAGGGTTTTATAAGAGGGGATTCTTCGTT
ACGAAAAAGAAATACGCTGTTATTGATGAAGAGGGCAAGATCACGACCCGTGGCC
TGGAAATTGTGCGCCGTGATTGGAGCGAAATTGCAAAAGAAACGCAAGCGCGTG
TGCTGGAAGCGCTGCTGAAGGACGGCGACGTCGAAAAAGCTGTGCGTATTGTTAA
AGAGGTCACCGAGAAGCTGAGCAAATACGAGGTCCCGCCAGAGAAATTGGTGAT
TCACGAACAGATTACGCGTGACCTGAAAGACTATAAGGCCACCGGTCCGCATGTC
GCAGTGGCGAAGCGCCTGGCGGCTCGCGGTGTGAAGATCCGTCCGGGTACCGTC
ATTAGCTATATCGTGCTGAAGGGCAGCGGTCGTATCGGCGACCGTGCGATTCCGTT
CGACGAATTTGATCCGACCAAACACAAATATGATGCGGAATACTATATTGAGAACC
AAGTGCTGCCAGCCGTTGAGCGTATTCTGCGCGCCTTCGGTTACCGCAAGGAAGA
TCTGCGTTACCAGAAAACTCGTCAGGTCGGTCTGTCCGCATGGCTGAAACCGAAG
GGCACCTGA
Nucleotide sequence of Pab DNA polymerase (SEQ ID NO: 6):
ATGATAATCGATGCTGATTACATAACGGAAGATGGCAAGCCGATAATAAGGATATTC
AAAAAGGAAAAGGGAGAGTTTAAGGTAGAATACGATAGGACGTTTAGACCCTACA
TTTATGCTCTTTTAAAGGATGATTCGGCCATAGATGAGGTTAAGAAGATAACCGCC
GAGAGGCACGGAAAGATAGTCAGGATAACCGAGGTTGAGAAAGTCCAGAAGAAA
TTCCTAGGAAGGCCAATAGAAGTCTGGAAGCTCTATCTTGAGCATCCCCAGGATGT
TCCAGCCATAAGAGAGAAGATAAGGGAACATCCAGCTGTAGTTGATATATTTGAAT
ACGACATACCCTTTGCGAAGCGCTACCTCATAGACAAGGGATTGACTCCAATGGA
GGGGAACGAGGAGCTAACGTTTCTAGCCGTTGATATAGAAACATTGTACCAT
GAAGGAGAGGAGTTCGGGAAAGGGCCAATAATAATGATCAGCTACGCCGAC
GAGGAAGGGGCCAAGGTGATAACTTGGAAGAGCATAGACTTACCTTACGTTG
AAGTGGTTTCGAGCGAGAGGGAGATGATAAAGAGGCTCGTGAAGGTAATTA
GAGAGAAAGATCCCGACGTGATAATAACGTACAATGGTGATAATTTCGACTTT
CCGTACCTCTTAAAGAGGGCTGAAAAGCTCGGAATAAAGCTCCCCCTTGGAA
GGGACAATAGCGAGCCGAAAATGCAGAGGATGGGGGATTCATTAGCCGTAGA
GATAAAGGGCAGAATACACTTCGATTTATTCCCCGTCATAAGAAGAACGATCA
ACCTTCCAACATACACCCTCGAAGCGGTTTATGAGGCTATATTTGGAAAGTCT
AAGGAGAAAGTCTATGCCCATGAGATAGCTGAGGCCTGGGAAACCGGGAAA
GGGCTAGAGAGGGTAGCTAAGTATTCAATGGAAGATGCGAAGGTAACCTTTG
AGCTCGGAAAGGAGTTCTTCCCGATGGAAGCCCAGCTAGCTAGGCTCGTTGG
CCAGCCAGTTTGGGACGTTTCAAGGTCGAGCACCGGAAACCTCGTTGAGTGGTTT
CTCCTTAGGAAGGCCTACGAGAGAAATGAGCTCGCGCCCAATAAACCGGACGAG
AGGGAATACGAGAGAAGGCTAAGAGAGAGCTATGAAGGGGGTTACGTTAAGGAG
CCAGAGAAGGGATTGTGGGAAGGGATAGTCAGCTTAGACTTTAGGTCCCTATATCC
CTCTATAATTATAACTCACAACGTCTCACCAGACACTTTGAATAGAGAAAATTGCA
AGGAATATGACGTTGCCCCCCAAGTGGGGCACAGATTCTGCAAGGATTTCCCAGG
ATTCATACCAAGCTTACTGGGTAACCTACTGGAGGAGAGACAAAAGATAAAA
AAGAGAATGAAAGAAAGTAAAGATCCCGTCGAGAAGAAACTCCTTGATTACA
GACAGAGAGCTATAAAAATACTTGCAAACAGCTATTATGGCTATTATGGATATG
CAAAGGCCAGATGGTACTGTAAAGAGTGTGCAGAGAGCGTAACCGCATGGGGAA
GGCAGTACATAGACCTGGTTAGGAGGGAACTTGAGAGCAGAGGATTTAAAGTTCT
CTACATAGACACAGATGGCCTCTACGCAACGATTCCTGGAGCCAAGCATGAGGAA
ATAAAAGAGAAGGCATTGAAGTTCGTCGAGTACATAAACTCCAAGTTACCTGGGC
TTCTTGAATTGGAATACGAAGGTTTCTACGCGAGAGGGTTCTTCGTGACGAAGAA
AAAGTACGCACTAATCGACGAGGAAGGAAAGATAGTTACGAGGGGGCTCGAAAT
AGTAAGGAGAGATTGGAGTGAAATAGCAAAGGAGACCCAGGCCAAGGTTCTTGA
GGCAATACTCAAGCACGGTAACGTTGATGAGGCCGTAAAAATAGTAAAGGAGGTT
ACAGAAAAACTCAGTAAATATGAAATACCACCCGAAAAGCTTGTAATTTATGAGCA
GATAACGAGGCCTCTGAGCGAGTATAAAGCGATAGGCCCTCACGTTGCAGTAGCT
AAAAGGCTCGCAGCGAAGGGAGTAAAAGTTAAGCCAGGGATGGTTATCGGTTAC
ATAGTTTTGAGGGGAGACGGGCCAATAAGCAAGAGGGCCATAGCTATAGAGGAGT
TCGATCCCAAAAAGCATAAGTACGATGCCGAATACTACATAGAGAACCAAGTTCTG
CCAGCGGTGGAGAGGATATTGAGAGCATTTGGTTATCGCAAGGAGGATTTGAAGT
ATCAAAAAACTAAACAAGTGGGCCTTGGAGCATGGCTTAAGTTCTGA
Nucleotide sequence of Pfu DNA polymerase (SEQ ID NO: 7):
ATGATTTTAGATGTGGATTACATAACTGAAGAAGGAAAACCTGTTATTAG
GCTATTCAAAAAAGAGAACGGAAAATTTAAGATAGAGCATGATAGAACTTTTAGA
CCATACATTTACGCTCTTCTCAGGGATGATTCAAAGATTGAAGAAGTTAAGAAAAT
AACGGGGGAAAGGCATGGAAAGATTGTGAGAATTGTTGATGTAGAGAAGGTTGA
GAAAAAGTTTCTCGGCAAGCCTATTACCGTGTGGAAACTTTATTTGGAACATCCCC
AAGATGTTCCCACTTTAAGAGAAAAAGTTAGAGAACATCCAGCAGTTGTGGACAT
CTTCGAATACGATATTCCATTTGCAAAGAGATACCTCATCGACAAAGGCCTAATACC
AATGGAGGGGGAAGAAGAGCTAAAGATTCTTGCCTTCGATATAGAAACCCTCTATC
ACGAAGGAGAAGAGTTTGGAAAAGGCCCAATTATAATGATTAGTTATGCAGATGA
AAATGAAGCAAGGGTGATTACTTGGAAAAACATAGATCTTCCATACGTTGAGTCA
GTATCAACCGAGAAAGAGATGATAAAGAGATTTCTCAGGATTATCAGGGAGAAGG
ATCCTGACATTATAGTTACTTATAATGGAGACTCATTCGACTTCCCATATTTAGCGAA
AAGGGCAGAAAAACTTGGGATTAAATTAACCATTGGAAGAGATGGAAGCGAGCC
CAAGATGCAGAGAATAGGCGATATGACGGCTGTAGAAGTCAAGGGAAGAATACAT
TTCGACTTGTATCATGTAATAAGGACAACAATAAATCTCCCAACATACACACTAGA
GGCTGTATATGAAGCAATTTTTGGAAAGCCAAAGGAGAAGGTATACGCCGACGAG
ATAGCAAAAGCCTGGGAAAGTGGAGAGAACCTTGAGAGAGTTGCCAAATACTCG
ATGGAAGATGCAAAGGCAACTTATGAACTCGGGAAAGAATTCCTTCCAATGGAAA
TTCAGCTTTCAAGATTAGTTGGACAACCTTTATGGGATGTTTCAAGGTCAAGCACA
GGGAACCTTGTAGAGTGGTTCTTACTTAGGAAAGCCTACGAAAGAAACGAAGTAG
CTCCAAACAAGCCAAGTGAAGAGGAGTATCAAAGAAGGCTCAGGGAGAGCTA
CACAGGTGGATTCGTTAAAGAGCCAGAAAAGGGGTTGTGGGAAAACATAGTA
TACCTAGATTACAAATCACTATATCCCTCGATTATAATTACCCACAATGTTTCT
CCCGATACTCTAAATCTTGAGGGATGCAAGAACTATGATATCGCTCCTCAAGT
AGGCCACAAGTTCTGCAAGGACATCCCTGGTTTTATACCAAGTCTCTTGGGACA
TTTGTTAGAGGAAAGACAAAAGATTAAGACAAAAATGAAGGAAACTCAAGATCC
TATAGAAAAAATACTCCTTGACTATAGACAAAAAGCGATAAAACTCTTAGCAAATT
CTTTCTACGGATATTATGGCTATGCAAAAGCAAGATGGTACTGTAAGGAGTGTG
CTGAGAGCGTTACTGCCTGGGGAAGAAAGTACATCGAGTTAGTATGGAAGGA
GCTCGAAGAAAAGTTTGGATTTAAAGTCCTCTACATTGACACTGATGGTCTCT
ATGCAACTATCCCAGGAGGAGAAAGTGAGGAAATAAAGAAAAAGGCTCTAGA
ATTTGTAAAATACATAAATTCAAAGCTCCCTGGACTGCTAGAGCTTGAATATG
AAGGGTTTTATAAGAGGGGATTCTTCGTTACGAAGAAGAGGTATGCAGTAATA
GATGAAGAAGGAAAAGTCATTACTCGTGGTTTAGAGATAGTTAGGAGAGATTGGA
GTGAAATTGCAAAAGAAACTCAAGCTAGAGTTTTGGAGACAATACTAAAACACGG
AGATGTTGAAGAAGCTGTGAGAATAGTAAAAGAAGTAATACAAAAGCTTGCCAAT
TATGAAATTCCACCAGAGAAGCTCGCAATATATGAGCAGATAACAAGACCATTACA
TGAGTATAAGGCGATAGGTCCTCACGTAGCTGTTGCAAAGAAACTAGCTGCTAAA
GGAGTTAAAATAAAGCCAGGAATGGTAATTGGATACATAGTACTTAGAGGCGATGG
TCCAATTAGCAATAGGGCAATTCTAGCTGAGGAATACGATCCCAAAAAGCACAAG
TATGACGCAGAATATTACATTGAGAACCAGGTTCTTCCAGCGGTACTTAGGATATTG
GAGGGATTTGGATACAGAAAGGAAGACCTCAGATACCAAAAGACAAGACAAGTC
GGCCTAACTTCCTGGCTTAACATTAAAAAATCCTGA
Nucleotide sequence of KOD DNA polymerase (SEQ ID NO: 8):
ATGATTCTGGACACCGATTACATCACCGAAGATGGCAAGCCAGTTA
TCCGCATTTTCAAAAAAGAGAATGGTGAATTCAAGATCGAATATGATCGTACC
TTCGAGCCGTACTTCTATGCTCTGCTGAAAGACGATAGCGCGATTGAGGAGG
TCAAGAAAATCACCGCGGAGCGTCACGGTACGGTTGTTACCGTGAAACGCG
TGGAGAAAGTCCAGAAGAAATTTCTGGGTCGCCCGGTTGAAGTGTGGAAGC
TGTACTTTACGCATCCGCAAGATGTTCCGGCGATTCGCGATAAGATTCGTGA
GCACCCGGCAGTCATTGACATCTACGAGTATGACATTCCGTTCGCCAAGCGT
TATCTGATCGATAAGGGTCTGGTCCCGATGGAGGGTGACGAAGAACTGAAGAT
GCTGGCGTTCGACATCGAAACTCTGTACCACGAGGGTGAAGAGTTTGCCGAGGGT
CCGATCTTGATGATTTCCTACGCGGACGAAGAGGGCGCACGTGTTATCACGTGGA
AAAATGTTGATCTGCCGTATGTTGACGTCGTAAGCACCGAGCGTGAGATGATCAA
ACGTTTTCTGCGCGTTGTTAAAGAAAAAGATCCTGACGTGCTGATCACCTACAAC
GGTGACAATTTCGATTTCGCGTACCTGAAGAAACGTTGCGAAAAACTGGGTATTA
ACTTCGCGCTGGGTCGCGATGGCTCTGAACCGAAGATCCAGCGCATGGGTGATCG
TTTTGCGGTCGAGGTGAAGGGTCGCATTCATTTCGACCTGTACCCGGTGATTCGTC
GTACCATCAACTTGCCGACTTACACCCTGGAAGCCGTCTATGAAGCTGTATTTGGT
CAACCGAAAGAAAAAGTGTACGCTGAGGAAATTACGACGGCGTGGGAAACCGGT
GAGAACCTGGAGCGCGTTGCACGTTATTCTATGGAGGACGCGAAAGTTACCTACG
AACTGGGTAAAGAGTTCCTGCCGATGGAGGCCCAACTGTCCCGTCTGATCGGCCA
AAGCCTGTGGGACGTTAGCCGCAGCAGCACCGGTAACTTAGTTGAATGGTTC
TTGCTGCGTAAGGCATACGAACGCAATGAGCTGGCGCCGAACAAACCGGAC
GAGAAAGAATTGGCGCGTCGCCGCCAGAGCTATGAGGGTGGTTATGTCAAAGAAC
CGGAGCGCGGCTTGTGGGAGAACATCGTCTATTTGGATTTTCGTAGCCTGTACCCG
AGCATCATTATCACGCATAATGTGAGCCCGGATACGTTGAATCGTGAGGGCTGTAA
GGAATACGACGTGGCGCCTCAGGTTGGCCACCGTTTCTGCAAGGACTTTCCGGGC
TTTATCCCGAGCCTGCTGGGTGATTTGCTGGAGGAACGTCAGAAAATCAAGAAGA
AGATGAAAGCAACCATTGATCCGATCGAGCGCAAATTACTGGACTACCGTCAACG
TGCCATCAAGATCCTGGCGAATTCGTATTATGGTTACTATGGCTACGCGCGTGCGCG
CTGGTATTGCAAAGAGTGTGCCGAGAGCGTGACCGCTTGGGGTCGTGAGTACATT
ACCATGACGATCAAAGAGATTGAAGAGAAATACGGCTTTAAGGTTATCTATAGCGA
CACCGACGGTTTCTTTGCAACTATCCCTGGCGCAGACGCAGAAACCGTTAAGAAA
AAGGCAATGGAGTTTCTGAAGTATATCAACGCGAAGTTGCCAGGCGCCCTGGAAC
TGGAGTACGAGGGCTTCTACAAGCGTGGCTTTTTCGTGACGAAAAAGAAATACG
CTGTTATTGATGAAGAGGGCAAGATCACGACCCGTGGCCTGGAAATTGTGCG
CCGTGATTGGAGCGAAATTGCAAAAGAAACGCAAGCGCGTGTGCTGGAAGC
GCTGCTGAAGGACGGCGACGTCGAAAAAGCTGTGCGTATTGTTAAAGAGGT
CACCGAGAAGCTGAGCAAATACGAGGTCCCGCCAGAGAAATTGGTGATTCAC
GAACAGATTACGCGTGACCTGAAAGACTATAAGGCCACCGGTCCGCATGTCG
CAGTGGCGAAGCGCCTGGCGGCTCGCGGTGTGAAGATCCGTCCGGGTACCG
TCATTAGCTATATCGTGCTGAAGGGCAGCGGTCGTATCGGCGACCGTGCGAT
TCCGTTCGACGAATTTGATCCGACCAAACACAAATATGATGCGGAATACTATA
TTGAGAACCAAGTGCTGCCAGCCGTTGAGCGTATTCTGCGCGCCTTCGGTTA
CCGCAAGGAAGATCTGCGTTACCAGAAAACTCGTCAGGTCGGTCTGTCCGC
ATGGCTGAAACCGAAGGGCACCTGA

It can be understood by those skilled in the art that the isolated nucleic acid has the similar features and advantages of the chimeric DNA polymerases as described above, which will not be repeated herein.

### Construct

In yet another aspect of the present disclosure, the present disclosure provides a construct. According to an embodiment of the present disclosure, the construct contains the isolated nucleic acid as described above. Thus, the construct according to the embodiment of the present disclosure can express the chimeric DNA polymerase having the properties such as high processivity, high elongation rate, thermal stability, strong resistance to salts, and high fidelity, which can meet the requirements of DNA amplification, synthesis, detection, sequencing, etc., thereby having a broad application prospect.

It will be understood by those skilled in the art that the construct has the similar features and advantages of the isolated nucleic acid as described above, which will not be repeated herein.

### Recombinant Cell or Recombinant Microorganism

In yet another aspect of the present disclosure, the present disclosure provides a recombinant cell or recombinant microorganism. According to an embodiment of the present disclosure, the recombinant cell or recombinant microorganism contains the isolated nucleic acid as described above. Thus, the recombinant cell or recombinant microorganism according to the embodiments of the present disclosure can express the chimeric DNA polymerase having the properties such as high processivity, high elongation rate, thermal stability, strong resistance to salts, and high fidelity, which can meet the requirements of DNA amplification, synthesis, detection, sequencing, etc., thereby having a broad application prospect.

Those skilled in the art can understand that the recombinant cell or recombinant microorganism has the similar features and advantages of the isolated nucleic acid as described above, which will not be repeated herein.

### Method for Obtaining Chimeric DNA Polymerase

In yet another aspect of the present disclosure, the present disclosure provides a method for obtaining the aforementioned chimeric DNA polymerase. According to an embodiment of the present disclosure, the method includes: culturing the aforementioned recombinant cell or recombinant microorganism under conditions suitable for expressing the chimeric DNA polymerase, so as to obtain the chimeric DNA polymerase. Thus, the method according to the method of the embodiment of the present disclosure can obtain the chimeric DNA polymerases having the properties such as high processivity, high elongation rate, thermal stability, strong resistance to salts, and high fidelity, which can meet the requirements of DNA amplification, synthesis, detection, sequencing, etc., thereby having a broad application prospect.

Those skilled in the art can understand that the method for obtaining the chimeric DNA polymerase has the similar features and advantages of the chimeric DNA polymerases as described above, which will not be repeated herein.

### Kit

In yet another aspect of the present disclosure, the present disclosure provides a kit. According to an embodiment of the present disclosure, the kit contains the aforementioned chimeric DNA polymerase, isolated nucleic acid, construct, recombinant cell or recombinant microorganism. Therefore, DNA amplification using the kit according to the embodiment of the present disclosure has the advantages such as high amplification product yield and high amplification accuracy, and thus the kit is suitable for wide production and application.

It should be noted that the kit of the present disclosure may further contain other reagents commonly used in the art, for example, buffers, primers, nucleoside triphosphates, etc., which can be flexibly selected according to the actual situation and are not specifically limited in the present disclosure.

It will be understood by those skilled in the art that the kit has the similar features and advantages of the chimeric DNA polymerases, the isolated nucleic acid, the construct, and the recombinant cell or recombinant microorganism, as described above, which will not be repeated herein.

### Use

In yet another aspect of the present disclosure, the present disclosure provides use of the aforementioned chimeric DNA polymerase, the isolated nucleic acid, the construct, the recombinant cell or recombinant microorganism, or the kit in DNA amplification. As a result, the DNA amplification has the advantages of a high amplification product yield and high amplification accuracy, and is suitable for wide production and application.

According to embodiments of the present disclosure, the chimeric DNA polymerase, the isolated nucleic acid, the construct, the recombinant cell or recombinant microorganism, or the kit can be used for genetic screening, sequencing or mutation detection. The above DNA polymerases, due to their properties such as high processivity, high elongation rate, thermal stability, strong resistance to salts, and high fidelity, can be effectively applied to gene screening, sequencing, or mutation detection, which has high requirements for the amplification yield and fidelity.

It will be understood by those skilled in the art that that the use has the similar features and advantages of the chimeric DNA polymerases, the isolated nucleic acid, the construct, the recombinant cell or recombinant microorganism, or the kit, as described above, which will not be repeated herein.

The solutions of the present disclosure will be explained below in conjunction with the examples. Those skilled in the art will understand that the following examples are only used to illustrate the present disclosure, and should not be construed as limiting the scope of the present disclosure. The specific technique or condition in the examples are those described in the literatures in the related field or the product specification, unless they are specifically indicated. The reagents or instruments without indicating the manufacturers are conventional products that can be obtained from the market.

### Example 1

### Design and Construction of Chimeric DNA Polymerases

The Pfu, Pab, and KOD DNA polymerases have different phenotypic characteristics. Specifically, among all the DNA polymerases having thermal stability and fidelity, the Pfu DNA polymerase has the lowest error probability, with an error probability of about 2.0×10⁻⁶. The Pab DNA polymerase also exhibits a low amplification probability and higher thermal stability. The KOD DNA polymerase, as a DNA polymerase with high amplification ability, has an amplification speed that is 2 times that of the Taq DNA polymerase and 6 times that of the Pfu DNA polymerase, and thus has a high amplification yield (about 300 nts).

The chimeric DNA polymerase in this example is a chimeric combination of the Pfu, Pab and KOD DNA polymerases (shown in FIG. 1). Specifically, nucleotide sequences (sites 1 to 390 and sites 1012 to 1119) in the N-end domain of the KOD DNA polymerase, a nucleotide sequence (sites 1771 to 2325) in the thumb domain of the KOD DNA polymerase, a nucleotide sequence (sites 391 to 1011) in the exonucleolytic domain of the Pab DNA polymerase, a sequence (sites 1345 to 1500) in the finger domain of the Pab DNA polymerase, and nucleotide sequences (sites 1120 to 1344 and sites 1501 to 1773) in the palm domain of the Pfu DNA polymerase were constructed between the XhoI/BamHI digestion sites of the prokaryotic expression vector pET28a. The vector carrying the above nucleotide sequences was transferred into Escherichia coli BL21 (DE3), followed by culturing to obtain the expression strains. The chimeric DNA polymerase exhibits high amplification capacity similar to the KOD DNA polymerase, and has a lower amplification mismatch probability than the KOD DNA polymerase.

### Example 2

### Fermentative Expression and Purification of Chimeric DNA Polymerase

1. Fermentation expression: the expression strains obtained in Example 1 were scaled up at a ratio of 1:100 and inoculated into a liquid LB medium containing kanamycin. The culture was shaken at 220 rpm and 37° ° C. until OD600=0.6, followed by adding 0.5 mM IPTG, and low temperature induction expression at 16° C. with shaking at 220 rpm overnight (16 h). The bacterial pellet was collected after centrifugation at 6000 rpm for 8 min.

2. Fermentation cell treatment: the bacteria were re-suspended with a bacteria re-suspending solution A (20 mM Tris, 300 mM NaCl, 20 mM Imidazole, 5% Glycerol, pH 8.0), at a mass-volume ratio of bacteria weight (g) to bacteria re-suspending solution (ml) being 1:20, followed by ultrasonication. The supernatant was collected after a centrifugation at 12,000 rpm for 20 min. After denaturation in a water bath at 75° C. for 30 min, the supernatant was collected after a centrifugation at 12,000 rpm for 20 min.

3. Ni column purification: the collected supernatant was filtered through a 0.22 μm filtering device. After the Ni column was washed and equilibrated with the bacteria re-suspending solution A, the above filtrate through the 0.22 μm filter was added to the column, and the column was gradient-eluted with adjusting a concentration of imidazole in the eluent (20 mM Tris, 300 mM NaCl, 5% Glycerol, 500 mM Imidazole, pH 8.0). The fractions from the column were collected, and the active fractions were analyzed by SDS-PAGE. The fractions of pure target proteins observed on a Coomassie-stained SDS-PAGE gel were pooled.

4. Anion column purification: the pooling sample of the above fractions was purified through an ion column to control endonuclease residues and nucleic acid residues in the sample. The pooling sample of fractions was dialyzed into Buffer C (20 mM Tris, 100 mM NaCl, 5% Glycerol, pH 8.0), the concentration of salt ions in Buffer D (20 mM Tris, 500 mM NaCl, 5% Glycerol, pH 8.0) was adjusted for gradient elution, and the collected elution column fraction was the novel chimeric DNA polymerase. The obtained sample was dialyzed to a storage system (20 mM Tris, 200 mM KCl, 50% Glycerol, 0.2 mM EDTA, 2 mM DTT, 0.001% Tween 20, 0.001% NP40, pH 8.0). The enzymes obtained from purification are illustrated in FIG. 2, and the target proteins with higher purity were obtained.

### Example 3: Amplification Performance of Chimeric DNA Polymerase

The chimeric DNA polymerases obtained in Example 2 of the present disclosure were amplified with λDNA as a template, and the amplified fragments have a length of 2 Kb to 8 Kb. Among them, the extension time of the Pfu DNA polymerase was 60 s/kb, and the extension time of the Pab, KOD and chimeric DNA polymerase was 30 s/kb.

The primer sequences are as follows:

lam-F:
(SEQ ID NO: 9)
CCTCTGTCGTTTCCTTTCTCTGTTTTTGTCCGTGG
lam2K-R:
(SEQ ID NO: 10)
CGTCTGTTCATCGTCGTGGCGGCCCATAATAATCT
lam4K-R:
(SEQ ID NO: 11)
GCACTCTTTCTCGTAGGTACTCAGTCCGGCTTCT
lam8K-R:
(SEQ ID NO: 12)
CGGGAATACGACGGTTACCCACCACAAGCACG

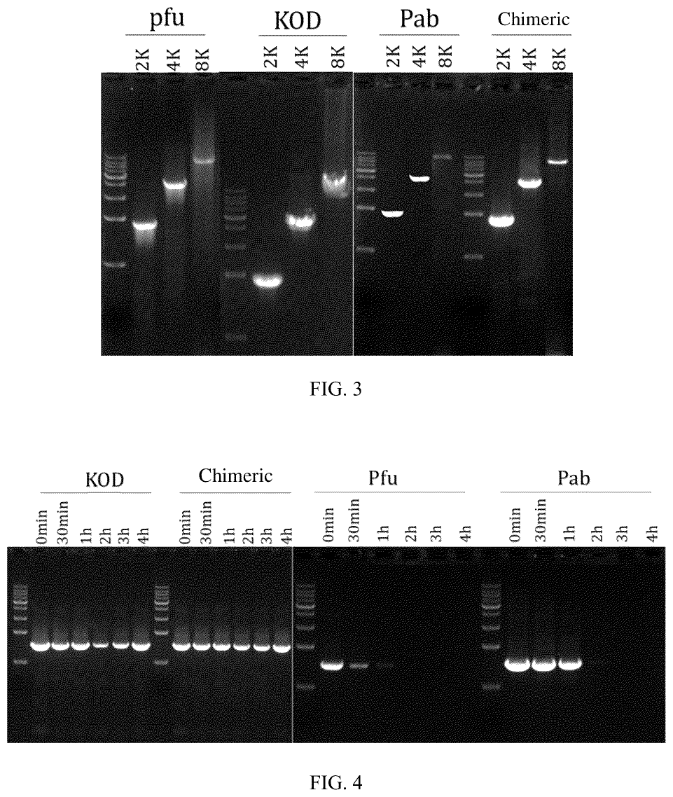

The amplification reaction procedure and system are shown in Table 2. The results are illustrated in FIG. 3. FIG. 3 indicates that the new chimeric DNA polymerase has a higher yield of the amplified target product and better amplification effect than the Pfu and Pab DNA polymerase; and that the amplification effect of the chimeric DNA polymerases was comparable with the amplification effect of the KOD DNA polymerase.

TABLE 2
Amplification reaction conditions

| Temper- | | Number of | | |
|---|---|---|---|---|
| ature | Time | cycles | Components | Volume (μl) |
| 94° C. | 3 min | 1 | 5× PCR Buffer | 5 |
| 95° C. | 20 sec | 30 | λDNA (10 ng/ul) | 1 |
| 60° C. | 15 sec | | Primer (10 uM) | 0.5 each |
| 72° C. | | | dNTPs (10 mM) | 0.4 |
| 72° C. | 5 min | 1 | Enzyme | 1 |
| 8° C. | ∞ | 1 | H₂O | Supplement to |
| | | | | 25 μl |

### Example 4

### Amplification Fidelity Analysis of Chimeric DNA Polymerases

LacIQZ a gene was amplified respectively with the Pab DNA polymerase, the Pfu DNA polymerase, the KOD DNA polymerase, and the chimeric DNA polymerase. The amplified fragments and the vectors were digested with XbaI/NcoI double enzymes. The amplified fragments and the vector fragments were respectively collected with gel and enzymatically ligated, and the ligation mixture was transformed into E. coli DH5a and the cells were inoculated on LB-Amp-X-gal plates for culture. The number of blue bacterial colonies, the number of white bacterial colonies and the total number of bacterial colonies were counted, to calculate the fidelity.

The sequences of the primers for amplification of the LacIQZ a gene are as follows:

Lac-F:
(SEQ ID NO: 13)
GTTTTCCCAGTCACGAC
Lac-R:
(SEQ ID NO: 14)
GGTATCTTTATAGTCCTGTCG

Equation for calculating fidelity:

$$Mismatch ratio = 1 {-}^{number of bases \times number of cycles} \sqrt{\frac{number of blue bacterial colonies}{total number of bacterial colonies}}$$

The results are shown in Table 3 below. The above results demonstrate that the chimeric DNA polymerase has improved amplification fidelity performance compared to the KOD DNA polymerase.

TABLE 3
Colony count and mismatch ratio

| | Number | Number | Total | |
|---|---|---|---|---|
| | of white | of blue | number of | Mismatch |
| | bacterial | bacterial | bacterial | ratio |
| | colonies | colonies | colonies | (×10⁻⁶) |
| Pfu | 183 | 9290 | 9473 | 1.41 |
| Kod | 472 | 7319 | 7791 | 4.51 |
| Pab | 157 | 8763 | 8920 | 1.28 |
| Chimeric DNA | 151 | 8105 | 8256 | 1.33 |
| polymerase | | | | |

### Example 5

### Thermal Stability Assay of Chimeric DNA Polymerase

The Pab, Pfu, KOD and chimeric polymerases were each incubated at 98° C. for 0, 30, 60, 90, 120 or 180 minutes. After the incubation, E. coli gDNA was amplified with each of the above polymerases, and the PCR products were analyzed by agarose gel.

The amplification primer sequences:

Ecoli-F:
(SEQ ID NO: 15)
AGAGTTTGATCMTGGCTCAG
Ecoli-R:
(SEQ ID NO: 16)
CGGTTACCTTGTTACGACTT

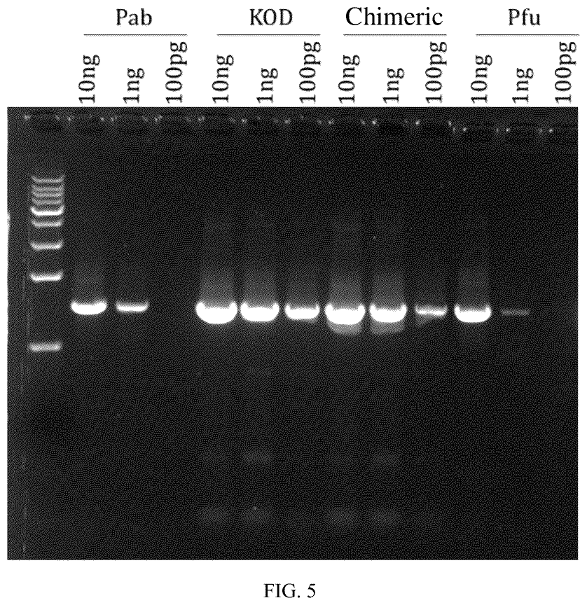

The amplification system and amplification procedure can refer to Example 3. The results are shown in FIG. 4.

The results showed that no amplified fragments were observed by the Pfu DNA polymerase after 2 h of pre-incubation at 98° C.; no amplified fragments were observed by the Pab DNA polymerase after 3 h of pre-incubation at 98° C.; and the PCR product was obtained through amplification by the chimeric DNA polymerase at all test time points.

### Example 6

### Amplification Using Chimeric DNA Polymerases Under the Condition of a Small Amount of Template

10 ng, 1 ng and 100 pg of E. coli gDNA were respectively added to the amplification system, and amplified with the Pfu, Pab, KOD and chimeric polymerases. The PCR products were analyzed by agarose gel. The amplification system and amplification procedure can refer to Example 3, and the sequences of the amplification primers can refer to Example 5. The results are shown in FIG. 5. With the three respective amounts of the added template, the target product can be obtained with a better yield through the amplification with the chimeric DNA polymerases.

In the specification, description with reference to the terms “an embodiment,” “some embodiments,” “example,” “specific example,” or “some examples”, etc., mean that specific features, structures, materials or characteristics described in connection with the embodiment or example are included in at least an embodiment or example of the present disclosure. In this specification, schematic representations of the above terms are not necessarily directed to the same embodiment or example. Furthermore, the particular features, structures, materials or characteristics described may be combined in any suitable manner in any one or more embodiments or examples. Furthermore, those skilled in the art may combine the different embodiments or examples as well as the features of the different embodiments or examples described in this specification, without conflicting each other.

Although the embodiments of the present disclosure have been illustrated and described above, it should be understood that the above embodiments are exemplary and should not be construed as limiting the present disclosure. Those skilled in the art can make changes, modifications, substitutions and variations to the embodiments within the scope of the present disclosure.

1. A chimeric DNA polymerase, comprising:
a first peptide fragment having at least 80% homology to at least one part of an amino acid sequence in an N-end domain of a KOD DNA polymerase;
a second peptide fragment having at least 80% homology to at least one part of an amino acid sequence in an exonucleolytic domain of a Pab DNA polymerase, an N-end of the second peptide fragment being connected to a C-end of the first peptide fragment;
a third peptide fragment having at least 80% homology to at least part of amino acids in the N-end domain of the KOD DNA polymerase, an N-end of the third peptide fragment being connected to a C-end of the second peptide fragment;
a fourth peptide fragment having at least 80% homology to at least part of amino acids in a palm domain of a Pfu DNA polymerase, an N-end of the fourth peptide fragment being connected to a C-end of the third peptide fragment;
a fifth peptide fragment having at least 80% homology to at least part of amino acids in a finger domain of the Pab DNA polymerase, an N-end of the fifth peptide fragment being connected to a C-end of the fourth peptide fragment;
a sixth peptide fragment having at least 80% homology to at least part of the amino acids in the palm domain of the Pfu DNA polymerase, an N-end of the sixth peptide fragment being connected to a C-end of the fifth peptide fragment; and
a seventh peptide fragment having at least 80% homology to at least part of amino acids in a thumb domain of the KOD DNA polymerase, an N-end of the seventh peptide fragment being connected to a C-end of the sixth peptide fragment.

2. The chimeric DNA polymerase according to claim 1, wherein the first peptide fragment has at least 80% homology to an amino acid sequence of sites 1 to 130 of the KOD DNA polymerase.

3. The chimeric DNA polymerase according to claim 1, wherein the second peptide fragment has at least 80% homology to an amino acid sequence of sites 131 to 337 of the Pab DNA polymerase.

4. The chimeric DNA polymerase according to claim 1, wherein the third peptide fragment has at least 80% homology to an amino acid sequence of sites 338 to 373 of the KOD DNA polymerase.

5. The chimeric DNA polymerase of claim 1, wherein the fourth peptide fragment has at least 80% homology to an amino acid sequence of sites 374 to 448 of the Pfu DNA polymerase.

6. The chimeric DNA polymerase of claim 1, wherein the fifth peptide fragment has at least 80% homology to an amino acid sequence of sites 449 to 500 of the Pab DNA polymerase.

7. The chimeric DNA polymerase of claim 1, wherein the sixth peptide fragment has at least 80% homology to an amino acid sequence of sites 501 to 591 of the Pfu DNA polymerase.

8. The chimeric DNA polymerase of claim 1, wherein the seventh peptide fragment has at least 80% homology to an amino acid sequence of sites 591 to 774 of the KOD DNA polymerase.

9. The chimeric DNA polymerase according to claim 1, wherein the chimeric DNA polymerase has an amino acid sequence set forth as SEQ ID NO: 1.

10. An isolated nucleic acid, encoding the chimeric DNA polymerase according to claim 1.

11. The isolated nucleic acid according to claim 10, wherein the isolated nucleic acid has a nucleotide sequence set forth as SEQ ID NO: 2.

12. A construct, comprising the isolated nucleic acid according to claim 10.

13. A recombinant cell or recombinant microorganism, containing the isolated nucleic acid according to claim 10.

14. A method for obtaining the chimeric DNA polymerase according to claim 1, comprising:
culturing a recombinant cell or recombinant microorganism containing an isolated nucleic acid encoding the chimeric DNA polymerase under conditions suitable for expressing the chimeric DNA polymerase to obtain the chimeric DNA polymerase.

15. A kit, containing the chimeric DNA polymerase according to claim 1.

16. The chimeric DNA polymerase according to claim 1, wherein the chimeric DNA polymerase is used in DNA amplification, genetic screening, sequencing, or mutation detection.

17. The isolated nucleic acid according to claim 10, wherein the isolated nucleic acid is used in DNA amplification, genetic screening, sequencing, or mutation detection.

18. The construct according to claim 12, wherein the construct is used in DNA amplification, genetic screening, sequencing, or mutation detection.

19. The recombinant cell or recombinant microorganism according to claim 13, wherein the recombinant cell or recombinant microorganism is used in DNA amplification, genetic screening, sequencing, or mutation detection.

20. The kit according to claim 15, wherein the kit is used in DNA amplification, genetic screening, sequencing, or mutation detection.


SEQUENCE LISTING (16 sequences)

SEQ ID NO: 1 - protein, 775 aa, Artificial Sequence
  synthesized
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPVIRRTINLPTYTLEAVYEAIFGKSKEKVYAHEIAEAWETGKGLERVAKYSMEDAKVTFELGKEFFPMEAQLARLVGQSLWDVSRSSTGNLVEWFLLRKAYERNELAPNKPDEEEYQRRLRESYTGGFVKEPEKGLWENIVYLDYKSLYPSIIITHNVSPDTLNLEGCKNYDIAPQVGHKFCKDIPGFIPSLLGNLLEERQKIKKRMKESKDPVEKKLLDYRQRAIKILANSYYGYYGYAKARWYCKECAESVTAWGRKYIELVWKELEEKFGFKVLYIDTDGLYATIPGGESEEIKKKALEFVKYINSKLPGLLELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 2 - DNA, 2328 bp, Artificial Sequence
  synthesized
atgattctggacaccgattacatcaccgaagatggcaagccagttatccgcattttcaaaaaagagaatggtgaattcaagatcgaatatgatcgtaccttcgagccgtacttctatgctctgctgaaagacgatagcgcgattgaggaggtcaagaaaatcaccgcggagcgtcacggtacggttgttaccgtgaaacgcgtggagaaagtccagaagaaatttctgggtcgcccggttgaagtgtggaagctgtactttacgcatccgcaagatgttccggcgattcgcgataagattcgtgagcacccggcagtcattgacatctacgagtatgacattccgttcgccaagcgttatctgatcgataagggtctggtcccgatggaggggaacgaggagctaacgtttctagccgttgatatagaaacattgtaccatgaaggagaggagttcgggaaagggccaataataatgatcagctacgccgacgaggaaggggccaaggtgataacttggaagagcatagacttaccttacgttgaagtggtttcgagcgagagggagatgataaagaggctcgtgaaggtaattagagagaaagatcccgacgtgataataacgtacaatggtgataatttcgactttccgtacctcttaaagagggctgaaaagctcggaataaagctcccccttggaagggacaatagcgagccgaaaatgcagaggatgggggattcattagccgtagagataaagggcagaatacacttcgatttattccccgtcataagaagaacgatcaaccttccaacatacaccctcgaagcggtttatgaggctatatttggaaagtctaaggagaaagtctatgcccatgagatagctgaggcctgggaaaccgggaaagggctagagagggtagctaagtattcaatggaagatgcgaaggtaacctttgagctcggaaaggagttcttcccgatggaagcccagctagctaggctcgttggccaaagcctgtgggacgttagccgcagcagcaccggtaacttagttgaatggttcttgctgcgtaaggcatacgaacgcaatgagctggcgccgaacaaaccggacgaagaggagtatcaaagaaggctcagggagagctacacaggtggattcgttaaagagccagaaaaggggttgtgggaaaacatagtatacctagattacaaatcactatatccctcgattataattacccacaatgtttctcccgatactctaaatcttgagggatgcaagaactatgatatcgctcctcaagtaggccacaagttctgcaaggacatccctggtttcataccaagcttactgggtaacctactggaggagagacaaaagataaaaaagagaatgaaagaaagtaaagatcccgtcgagaagaaactccttgattacagacagagagctataaaaatacttgcaaacagctattatggctattatggatatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaaaaagaaatacgctgttattgatgaagagggcaagatcacgacccgtggcctggaaattgtgcgccgtgattggagcgaaattgcaaaagaaacgcaagcgcgtgtgctggaagcgctgctgaaggacggcgacgtcgaaaaagctgtgcgtattgttaaagaggtcaccgagaagctgagcaaatacgaggtcccgccagagaaattggtgattcacgaacagattacgcgtgacctgaaagactataaggccaccggtccgcatgtcgcagtggcgaagcgcctggcggctcgcggtgtgaagatccgtccgggtaccgtcattagctatatcgtgctgaagggcagcggtcgtatcggcgaccgtgcgattccgttcgacgaatttgatccgaccaaacacaaatatgatgcggaatactatattgagaaccaagtgctgccagccgttgagcgtattctgcgcgccttcggttaccgcaaggaagatctgcgttaccagaaaactcgtcaggtcggtctgtccgcatggctgaaaccgaagggcacctga

SEQ ID NO: 3 - protein, 771 aa, Artificial Sequence
  synthesized
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPVIRRTINLPTYTLEAVYEAIFGKSKEKVYAHEIAEAWETGKGLERVAKYSMEDAKVTFELGKEFFPMEAQLARLVGQPVWDVSRSSTGNLVEWFLLRKAYERNELAPNKPDEREYERRLRESYEGGYVKEPEKGLWEGIVSLDFRSLYPSIIITHNVSPDTLNRENCKEYDVAPQVGHRFCKDFPGFIPSLLGNLLEERQKIKKRMKESKDPVEKKLLDYRQRAIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLKYQKTKQVGLGAWLKF

SEQ ID NO: 4 - protein, 775 aa, Artificial Sequence
  synthesized
MILDVDYITEEGKPVIRLFKKENGKFKIEHDRTFRPYIYALLRDDSKIEEVKKITGERHGKIVRIVDVEKVEKKFLGKPITVWKLYLEHPQDVPTLREKVREHPAVVDIFEYDIPFAKRYLIDKGLIPMEGEEELKILAFDIETLYHEGEEFGKGPIIMISYADENEARVITWKNIDLPYVESVSTEKEMIKRFLRIIREKDPDIIVTYNGDSFDFPYLAKRAEKLGIKLTIGRDGSEPKMQRIGDMTAVEVKGRIHFDLYHVIRTTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDYKSLYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 5 - protein, 774 aa, Artificial Sequence
  synthesized
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 6 - DNA, 2316 bp, Artificial Sequence
  synthesized
atgataatcgatgctgattacataacggaagatggcaagccgataataaggatattcaaaaaggaaaagggagagtttaaggtagaatacgataggacgtttagaccctacatttatgctcttttaaaggatgattcggccatagatgaggttaagaagataaccgccgagaggcacggaaagatagtcaggataaccgaggttgagaaagtccagaagaaattcctaggaaggccaatagaagtctggaagctctatcttgagcatccccaggatgttccagccataagagagaagataagggaacatccagctgtagttgatatatttgaatacgacataccctttgcgaagcgctacctcatagacaagggattgactccaatggaggggaacgaggagctaacgtttctagccgttgatatagaaacattgtaccatgaaggagaggagttcgggaaagggccaataataatgatcagctacgccgacgaggaaggggccaaggtgataacttggaagagcatagacttaccttacgttgaagtggtttcgagcgagagggagatgataaagaggctcgtgaaggtaattagagagaaagatcccgacgtgataataacgtacaatggtgataatttcgactttccgtacctcttaaagagggctgaaaagctcggaataaagctcccccttggaagggacaatagcgagccgaaaatgcagaggatgggggattcattagccgtagagataaagggcagaatacacttcgatttattccccgtcataagaagaacgatcaaccttccaacatacaccctcgaagcggtttatgaggctatatttggaaagtctaaggagaaagtctatgcccatgagatagctgaggcctgggaaaccgggaaagggctagagagggtagctaagtattcaatggaagatgcgaaggtaacctttgagctcggaaaggagttcttcccgatggaagcccagctagctaggctcgttggccagccagtttgggacgtttcaaggtcgagcaccggaaacctcgttgagtggtttctccttaggaaggcctacgagagaaatgagctcgcgcccaataaaccggacgagagggaatacgagagaaggctaagagagagctatgaagggggttacgttaaggagccagagaagggattgtgggaagggatagtcagcttagactttaggtccctatatccctctataattataactcacaacgtctcaccagacactttgaatagagaaaattgcaaggaatatgacgttgccccccaagtggggcacagattctgcaaggatttcccaggattcataccaagcttactgggtaacctactggaggagagacaaaagataaaaaagagaatgaaagaaagtaaagatcccgtcgagaagaaactccttgattacagacagagagctataaaaatacttgcaaacagctattatggctattatggatatgcaaaggccagatggtactgtaaagagtgtgcagagagcgtaaccgcatggggaaggcagtacatagacctggttaggagggaacttgagagcagaggatttaaagttctctacatagacacagatggcctctacgcaacgattcctggagccaagcatgaggaaataaaagagaaggcattgaagttcgtcgagtacataaactccaagttacctgggcttcttgaattggaatacgaaggtttctacgcgagagggttcttcgtgacgaagaaaaagtacgcactaatcgacgaggaaggaaagatagttacgagggggctcgaaatagtaaggagagattggagtgaaatagcaaaggagacccaggccaaggttcttgaggcaatactcaagcacggtaacgttgatgaggccgtaaaaatagtaaaggaggttacagaaaaactcagtaaatatgaaataccacccgaaaagcttgtaatttatgagcagataacgaggcctctgagcgagtataaagcgataggccctcacgttgcagtagctaaaaggctcgcagcgaagggagtaaaagttaagccagggatggttatcggttacatagttttgaggggagacgggccaataagcaagagggccatagctatagaggagttcgatcccaaaaagcataagtacgatgccgaatactacatagagaaccaagttctgccagcggtggagaggatattgagagcatttggttatcgcaaggaggatttgaagtatcaaaaaactaaacaagtgggccttggagcatggcttaagttctga

SEQ ID NO: 7 - DNA, 2328 bp, Artificial Sequence
  synthesized
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatgttcccactttaagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaagggtgattacttggaaaaacatagatcttccatacgttgagtcagtatcaaccgagaaagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataaggacaacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattacaaatcactatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatcctga

SEQ ID NO: 8 - DNA, 2325 bp, Artificial Sequence
  synthesized
atgattctggacaccgattacatcaccgaagatggcaagccagttatccgcattttcaaaaaagagaatggtgaattcaagatcgaatatgatcgtaccttcgagccgtacttctatgctctgctgaaagacgatagcgcgattgaggaggtcaagaaaatcaccgcggagcgtcacggtacggttgttaccgtgaaacgcgtggagaaagtccagaagaaatttctgggtcgcccggttgaagtgtggaagctgtactttacgcatccgcaagatgttccggcgattcgcgataagattcgtgagcacccggcagtcattgacatctacgagtatgacattccgttcgccaagcgttatctgatcgataagggtctggtcccgatggagggtgacgaagaactgaagatgctggcgttcgacatcgaaactctgtaccacgagggtgaagagtttgccgagggtccgatcttgatgatttcctacgcggacgaagagggcgcacgtgttatcacgtggaaaaatgttgatctgccgtatgttgacgtcgtaagcaccgagcgtgagatgatcaaacgttttctgcgcgttgttaaagaaaaagatcctgacgtgctgatcacctacaacggtgacaatttcgatttcgcgtacctgaagaaacgttgcgaaaaactgggtattaacttcgcgctgggtcgcgatggctctgaaccgaagatccagcgcatgggtgatcgttttgcggtcgaggtgaagggtcgcattcatttcgacctgtacccggtgattcgtcgtaccatcaacttgccgacttacaccctggaagccgtctatgaagctgtatttggtcaaccgaaagaaaaagtgtacgctgaggaaattacgacggcgtgggaaaccggtgagaacctggagcgcgttgcacgttattctatggaggacgcgaaagttacctacgaactgggtaaagagttcctgccgatggaggcccaactgtcccgtctgatcggccaaagcctgtgggacgttagccgcagcagcaccggtaacttagttgaatggttcttgctgcgtaaggcatacgaacgcaatgagctggcgccgaacaaaccggacgagaaagaattggcgcgtcgccgccagagctatgagggtggttatgtcaaagaaccggagcgcggcttgtgggagaacatcgtctatttggattttcgtagcctgtacccgagcatcattatcacgcataatgtgagcccggatacgttgaatcgtgagggctgtaaggaatacgacgtggcgcctcaggttggccaccgtttctgcaaggactttccgggctttatcccgagcctgctgggtgatttgctggaggaacgtcagaaaatcaagaagaagatgaaagcaaccattgatccgatcgagcgcaaattactggactaccgtcaacgtgccatcaagatcctggcgaattcgtattatggttactatggctacgcgcgtgcgcgctggtattgcaaagagtgtgccgagagcgtgaccgcttggggtcgtgagtacattaccatgacgatcaaagagattgaagagaaatacggctttaaggttatctatagcgacaccgacggtttctttgcaactatccctggcgcagacgcagaaaccgttaagaaaaaggcaatggagtttctgaagtatatcaacgcgaagttgccaggcgccctggaactggagtacgagggcttctacaagcgtggctttttcgtgacgaaaaagaaatacgctgttattgatgaagagggcaagatcacgacccgtggcctggaaattgtgcgccgtgattggagcgaaattgcaaaagaaacgcaagcgcgtgtgctggaagcgctgctgaaggacggcgacgtcgaaaaagctgtgcgtattgttaaagaggtcaccgagaagctgagcaaatacgaggtcccgccagagaaattggtgattcacgaacagattacgcgtgacctgaaagactataaggccaccggtccgcatgtcgcagtggcgaagcgcctggcggctcgcggtgtgaagatccgtccgggtaccgtcattagctatatcgtgctgaagggcagcggtcgtatcggcgaccgtgcgattccgttcgacgaatttgatccgaccaaacacaaatatgatgcggaatactatattgagaaccaagtgctgccagccgttgagcgtattctgcgcgccttcggttaccgcaaggaagatctgcgttaccagaaaactcgtcaggtcggtctgtccgcatggctgaaaccgaagggcacctga

SEQ ID NO: 9 - DNA, 35 bp, Artificial Sequence
  synthesized
cctctgtcgtttcctttctctgtttttgtccgtgg

SEQ ID NO: 10 - DNA, 35 bp, Artificial Sequence
  synthesized
cgtctgttcatcgtcgtggcggcccataataatct

SEQ ID NO: 11 - DNA, 34 bp, Artificial Sequence
  synthesized
gcactctttctcgtaggtactcagtccggcttct

SEQ ID NO: 12 - DNA, 32 bp, Artificial Sequence
  synthesized
cgggaatacgacggttacccaccacaagcacg

SEQ ID NO: 13 - DNA, 17 bp, Artificial Sequence
  synthesized
gttttcccagtcacgac

SEQ ID NO: 14 - DNA, 21 bp, Artificial Sequence
  synthesized
ggtatctttatagtcctgtcg

SEQ ID NO: 15 - DNA, 20 bp, Artificial Sequence
  synthesized
agagtttgatcmtggctcag

SEQ ID NO: 16 - DNA, 20 bp, Artificial Sequence
  synthesized
cggttaccttgttacgactt